(19) United States
(12) Patent Application Publication
(10) Pub. No.: US 2005/0245920 A1
(43) Pub. Date: Nov. 3, 2005

(54) Cell necrosis apparatus with cooled microwave antenna

(76) Inventors: Jeffrey M. Vitullo, Pottstown, PA (US); Michael R. Harding, Reading, PA (US); Richard L. Harding, Reinholds, PA (US)

Correspondence Address: Attn: Kenneth P. George, Esq.;Amster, Rothstein & Ebenstein LLP, 90 Park Avenue, New York, NY, 10016, US

(21) Appl. No.: 10/835,725

(22) Filed: Apr. 30, 2004

(51) Int. Cl. A61B 18/18

(52) U.S. Cl. 606/33; 607/156

(57) ABSTRACT

A cell necrosis apparatus for delivering thermal microwave energy to a specific site in a body, including: a. a microwave generator, b. a coolant delivery system for delivering and circulating a quantity of cooled liquid coolant via inlet and return passageways, c. a probe including a probe handle and a probe body having a proximal portion coupled to the probe handle and a distal portion, d. a microwave antenna in the distal portion of the probe body for applying thermal microwave energy to a specific site in cell necrosis treatment, and e. a microwave transmission line extending from the microwave generator to and through the probe handle and to and through the probe body to the microwave antenna and electrically coupled thereto, where the inlet and return coolant flow passageways extend from the coolant delivery system to and through the probe handle and thence extend coaxially about the microwave transmission line and along the length thereof within the probe body, and extend coaxially about the antenna and long the length thereof within the probe body, and where a first of the inlet and return coolant flow passageways is radially outward of and immediately adjacent the microwave transmission line and the antenna within the probe body and the other of the inlet flow passageways is radially outward of the first flow passageway.

### FIELD OF THE INVENTION

The present invention is in the field of microwave thermal therapy of tissue in a body and more particularly is an apparatus and method for treating interior tissue regions with a cell necrosis probe energized by a microwave generator.

### BACKGROUND OF THE INVENTION

Cell necrosis apparatus and methods are known in treatment of a variety of organs including but not limited to the prostate, liver, lungs, breasts and kidneys. In regard to prostate treatment, for example, U.S. Pat. No. 5,301,687 to Wong discloses a method and apparatus using an intracavity approach to the prostate through either the rectum or the urethra.

U.S. Pat. No. 5,273,886 to Edwards discloses an RF tissue heating system which includes a cooling feature where liquid coolant is used to cool the tissue being treated. More specifically sterile coolant water is applied to mucosal tissue at the targeted tissue region and subsequently is aspirated.

U.S. Pat. No. 5,733,319 to Nielson discloses apparatus for trans-urethral microwave thermal therapy of tissue which uses coolant passages arranged in an asymmetrical pattern in the antenna. This produces a corresponding asymmetrical pattern of radiation, which effectively focuses the microwave energy more on one side toward the tissue being treated and less on the opposite side toward tissue not intended for treatment. This cooling configuration further focuses the radiation by concentrating microwave emission at a given length of the antenna rather than spreading it up and down the antenna length.

The Nielson apparatus further includes a coolant-sensor interface module for sensing the temperature and pressure of the circulating liquid coolant. This interface module is relatively complex as is it constructed to prevent the circulating liquid from physically contacting the sensor control unit, which thus avoids a need for time consuming and expensive cleaning and sterilization of the sensor control unit between patents. The three above-referenced prior art patents are incorporated herein by reference.

The new invention is an improved cell necrosis apparatus and method which provide a number of advantages over known prior art systems.

### SUMMARY OF THE NEW INVENTION

In a first preferred embodiment the new invention includes a microwave generator, coolant circulating system, a flexible cooled transmission line, a probe handle connected to a probe body with its included antenna assembly, and a sharp probe tip for piercing the skin or other body tissue to initiate a percutaneous cell necrosis procedure. A particular aspect of the novelty herein is the configuration of the probe handle and probe body and antenna within the probe body to include a coaxial feedline cable and coaxial coolant entry and exit or return passageways in the probe body, thus establishing a symmetrical structure and a symmetrical radiation pattern emanating from the antenna. In this embodiment a fixed quantity of coolant from a reservoir is circulated by a pump, and temperature and pressure sensors in the probe for the coolant are absent and unneeded, as explained below. The present invention, furthermore, includes the method of cooling a microwave generating cell necrosis apparatus and a method of cell necrosis treatment with a microwave generating probe.

Within the microwave generator system the microwave generator, which generates a frequency of either 915 MHz or 2450 MHz, is connected to the proximal portion of a microwave transmission line whose distal end is coupled to a microwave antenna. The length of the radiating antenna assembly section will differ depending on the operating frequency. The generator should have an input power between 30 200 Watts. More than one output channel could be designed into the generator, so that simultaneous necrosis sites could be generated with multiple devices. The refrigerated coolant and pump system can either be integral to the microwave generator or self standing. Chilled water maintained at 40° F. and at a flow rate between 60 170 cc/min is preferred. By surface cooling the antenna radiating elements, the treatment volume may be extended to larger values of radii.

Examples of hypothetical clinical scenarios are as follows:

A. To create a 4.0 cm necrosis diameter within the patient's liver tumor site, the settings for the probe would be as follows: 200 watts of input power at 2450 MHz with a flow rate of 170 cc/min of 40° F. water. The probe would be placed accordingly within the tumor mass and operated for a total of 15 minutes. This would create a necrosis of at least 5.0 cm in diameter and 5.5 cm in length, and

B. To create a 2.0 cm necrosis lesion diameter within the patient's liver tumor site, the settings for the probe would be as follows: 200 watts of input power at 2450 MHz with a flow rate of 170 cc/min of 40° F. water. The probe would be placed accordingly within each tumor mass and operated simultaneously for a total of 10 minutes. This would create a necrosis of at least 3.0 cm in diameter and 3.5 cm in length.

Obviously, within the scope of this invention, parameters listed above and other parameters may be varied to achieve desired volumetric tissue heating and ablation.

From the microwave generator and coolant circulation pump extends a flexible coaxial feedline cable with coaxial coolant inlet and outlet passageways, whose distal end is coupled to the probe handle having coaxial lumens corresponding to those of the feedline cable. In the preferred embodiment the probe comprises a handle, a body, an antenna assembly, a tip and a temperature sensor at the tip.

The handle is used to manipulate the probe during the cell necrosis procedure. The handle also provides a communication link for the flexible transmission line and the coolant passageways with the corresponding elements in the probe body.

The purpose of the probe body is to contain and protect the distal portion of the cable chassis which includes the antenna radiating element. Depending on the type of procedure, insertion forces of different magnitudes are applied to the probe. For instance in percutaneous procedures, the probe must be capable of penetrating the patient's skin as well as the other body tissues that are in the insertion pathway to the tumor. With open and laparoscopic procedures these insertion forces are significantly less, since the organ of the patient is typically exposed and the probe can be directly placed.

The probe body consists of two single lumen tubes, which create a coaxial system. The outer lumen tube, which has a circular profile, is constructed of a plastic and metal composite. Non metallic materials such as plastic must be used to create the radiating element segment since metal in the field of the radiating element will disrupt the radiation pattern generated by the microwave antenna. This would result in uncontrollable and ineffective cell necrosis. The proximal portion of the outer lumen tube is constructed primarily of metal in order to minimize the potential for buckling of the probe shaft during insertion. Roughly 3.0 cm of the distal portion of the outer lumen is constructed of plastic in order to cover the 2.5 cm active length of the microwave antenna.

The inner lumen is constructed entirely of plastic and resides within the outer lumen. Plastics with high flexural moduli such as PEEK (Polyetheretherketone) and PEI (Polyetherimide) are optimal. Various fillers can be added to these resins in order to enhance their flexural moduli. The profile of the inner tube lumen is circular. However, a splined profile can further enhance the overall rigidity and integrity of the probe body. The splines reduce the flexure of the body during loading. To further reduce drag, the metallic portion of the outer lumens surface can be coated with a non stick surface such as PTFE.

At the distal end of the probe body, a tip is grafted to the outer lumen. To further minimize the insertion force of the probe, various tip designs can be used. These tips can be constructed of either plastic or metal. A metal edge at the distal end of the tip allows for a sharper and harder penetrating surface thereby minimizing the insertion force.

At the proximal end of the probe body a hub exists which supports both the inner and outer lumens. This hub is mounted to the probe handle. It should be noted that the construction of the probe body is not limited to a metal and plastic composite. Other variations exist. Alternatively, the probe body can be constructed entirely of plastic or high performance carbon fiber tubing.

The antenna assembly consists of the flexible coaxial feedline cable leading to the probe handle, a hermetically sealed splice or junction within the probe handle, a semi-rigid feedline cable and a radiating element within the probe body. The seal is both air tight and water tight so that coolant can be applied over the entire antenna assembly without causing an electrical short. Typically, electrical characteristics for each of these cables include excellent power handling and insertion loss properties. Calculations for antenna output are set forth later herein in the description of the preferred embodiment.

The antenna consists of conventional proximal, central and distal elements, but with changed and additional new construction for coaxial coolant lumens and an entire coolant delivery and control system.

The semi rigid coaxial cable is coated with a material of low dielectric property such as PTFE, which coating can be applied via means of spray coating or can be in the form of heat-shrink tubing. The antenna comprises proximal and distal elements joined to a central element positioned between them. The inner conductor of the transmission cable terminates at the distal element of the antenna. The proximal element of the antenna communicates with the distal end of the semi rigid cable's outer conductor. This junction is formed by means of welding the outer conductor of the semi rigid coaxial cable to the proximal element. Both the proximal and distal elements are constructed of medical grade metal. The central element is constructed of either epoxy or PTFE filler. The entire assembly is insulated with PTFE shrink tubing.

The coolant system circulates chilled water from the generator throughout the entire probe assembly for several reasons. First, chilling allows the antenna and transmission line to operate at higher powers over an extended period of time. Larger burn profiles result at higher wattages along with more time. Second, chilling of the antenna portion allows for a greater depth of penetration. By cooling the outer probe surface around the antenna, the therapeutic heating radius is increased. This is based on maximizing the coolant power in order to minimize the overall power difference of the system. This is more fully described later in the Description of the Preferred Embodiment. Third, lesions created by microwave antennas typically yield tear drop profiles resulting in tracking. This is caused by conductive energy, which tracks proximally beyond the antenna. Cooling eliminates this profile and allows for a more elliptical to spherical lesion with no tracking. Fourth, cooling of the probe body allows for patient comfort during the procedure at the entry site. This is extremely important during percutaneous procedures. It also allows the practitioner to hold the feedline during the procedure. All of these design features translate into large, controllable lesions.

An additional feature of the probe is a temperature sensor (either a thermocouple or a fiber optic sensor) is placed at the distal portion of the probe. The sensor is attached by means of an adhesive and runs proximally along the inner lumen to the connector. Temperature measuring is normally conducted only when the microwave system along with the pump is off. Temperature sensing is possible during the ablation process with more advanced measuring techniques; however, the information may be less useful. The purpose of the temperature sensing is twofold. First, it allows the practitioner to determine whether the device is working. Second, it allows the physician to re guide the probe into an untreated area by a simple temperature measurement.

In use of this new cell necrosis apparatus, the probe body is inserted into the tumor mass by means of advancing the probe handle. Through ultrasound or CT guidance, the practitioner can place the probe's radiating element into the tumor mass. Placement can be performed via an open surgery, laparoscopic or percutaneous procedure.

In preferred embodiments the diameter of the probe body can range from 7 to 9 Fr in size. For smaller tumor masses the size of the probe body can be reduced. Various exposure lengths can be constructed. A typical probe would be 8 Fr in diameter with an exposed length (distance from hub to the tip of the probe) of 20 cm. The probe handle is to be held by the practitioner during placement. A microwave antenna, housed within the distal tip of the probe body, has a 2.5 cm active length. The transmission line cable is roughly 7 ft long, which length allows the microwave generator system to be at a satisfactory distance away from the patient's bedside, considering space restrictions within procedural rooms. The length also allows the practitioner freedom to move the probe without obstruction during placement. The probe at its proximal end has a connector for attachment to a compatible microwave generator with an integral coolant system. Chilled water is circulated throughout the entire probe assembly. A temperature sensor is placed at the distal tip for temperature monitoring after necrosis. The entire probe is disposable.

An objective of this invention is to cool a probe of a cell necrosis apparatus by flowing coolant along the length of the antenna's outer surface via a passageway that circumferentially surrounds said surface. It is a further objective to establish by inner and outer concentric lumens, an inner annular flow path that surrounds the outer surface of the antenna and an outer flow path radially outward of and generally surrounding the inner flow path. In this embodiment the coolant from a source initially flows in the outer flow path in a distal direction lengthwise of the antenna, and then reverses direction and flows in the proximal direction along the surface of the antenna to produce a generally symmetric radiation pattern. It is a further objective to measure the temperature in the region of the distal end of the antenna, preferably when the antenna is inactive, and to thereafter adjust the coolant temperature and/or flow rate as needed. In establishing said coolant flow passageways it is an additional object to electrically insulate the antenna from the coolant flow. An additional objective is to cool via these coaxial flow passageways the microwave feedline within the probe body and within the transmission cable extending between the microwave generator and the probe handle.

In one preferred embodiment the present invention is a cell necrosis apparatus for delivering thermal microwave energy to a specific site in a body, comprising:
a. a microwave generator,
b. a coolant delivery system for delivering and circulating a quantity of cooled liquid coolant via inlet and return passageways,
c. a probe comprising a probe handle and a probe body having a proximal portion coupled to said probe handle and a distal portion,
d. a microwave antenna in said distal portion of said probe body for applying thermal microwave energy to said specific site in cell necrosis treatment, and
e. a microwave transmission line extending from said microwave generator to and through said probe handle and to and through said probe body to said microwave antenna and electrically coupled thereto,
f. said inlet and return coolant flow passageways extending from said coolant delivery system to and through said probe handle and thence extending coaxially about said microwave transmission line and along the length thereof within said probe body, and extending coaxially about said antenna and along the length thereof within said probe body, where a first of said inlet and return coolant flow passageways is radially outward of and immediately adjacent said microwave transmission line and said antenna within said probe body and the other of said inlet flow passageways being radially outward of said first flow passageway.

A further embodiment of this invention is a method of cell necrosis treatment with a cell necrosis apparatus including a microwave antenna in a probe, comprising the steps:
a. providing an annular coolant passageway adjacent and circumferentially surrounding said antenna and extending lengthwise and coaxial therewith,
b. providing a source of coolant, inlet and return duct means for communicating said coolant between said source and said antenna, and means for circulating said coolant through said inlet and return duct means, and
c. flowing said coolant in said annular coolant passageway while activating said microwave antenna, thereby producing a symmetrical radiation pattern about said antenna.

Features and advantages of the preferred embodiments of this invention are set forth in the following description and drawings, as well as in the appended claims.

### BRIEF DESCRIPTION OF THE DRAWINGS

FIG. 1 is a top, front perspective view of the new cell necrosis apparatus including the microwave probe,

FIG. 2 is a schematic perspective view of a probe placement into a tumor mass,

FIG. 3 is an elevational view of the probe body including a portion of the probe handle,

FIG. 3A is a sectional view taken along line 3A-3A in FIG. 3,

FIG. 3B is an elevational view similar to FIG. 3,

FIG. 3C is a sectional view taken along line 3C-3C in FIG. 3B,

FIG. 3D is a sectional view taken along line 3D-3D in FIG. 3B,

FIG. 3E is a sectional view taken along line 3E-3D in FIG. 3B,

FIG. 3F is a sectional view similar to FIG. 3E showing a different embodiment of the probe body,

FIG. 4 is an elevational view in section of the probe handle and coolant pathways therein,

FIG. 5 is an elevational view of a tip of the probe,

FIG. 5A is a front end view of the tip of FIG. 5,

FIG. 6 is an elevational view similar to FIG. 5a of an alternate tip,

FIG. 6A is a front end view of the tip of FIG. 6,

FIG. 7 is a top, front perspective schematic view of the cable chassis,

FIG. 8 is an elevational view of the microwave antenna,

FIG. 9 is an enlarged sectional, elevational view of the antenna of FIG. 8, and

FIG. 10 is an elevation view of the probe body showing the coolant passageways and the temperature sensor of the handle tip.

### DETAILED DESCRIPTION OF THE PREFERRED EMBODIMENTS

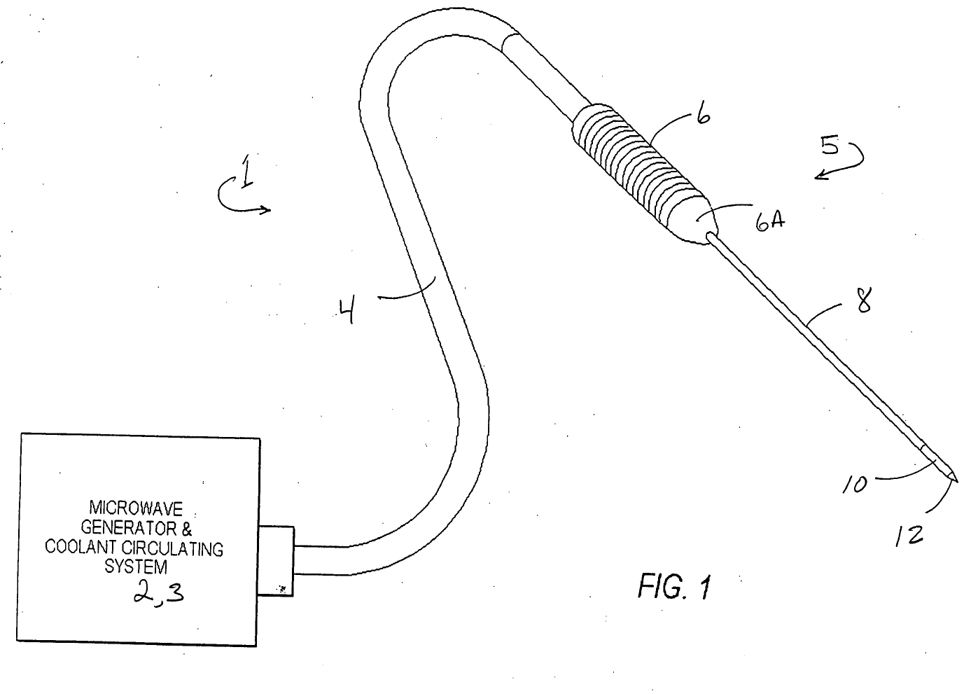

A preferred embodiment of the new cell necrosis apparatus 1 is seen in FIG. 1 as a combination of a microwave generator 2 and coolant reservoir and circulating apparatus 3, flexible transmission cable 4 including transmission line 18 therein, and probe 5, the probe consisting of probe handle 6, probe body 8, radiating element segment 10 at the distal part of the probe body, and probe tip 12 at the distal end of the probe body. The coolant passageways are configured to flow coolant coaxially in the probe body and about the antenna to produce a symmetrical radiation pattern.

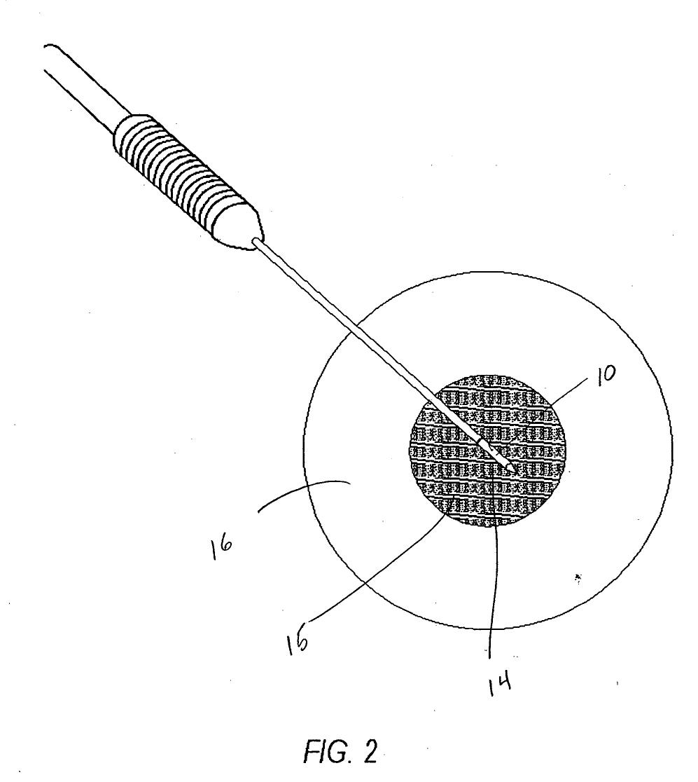

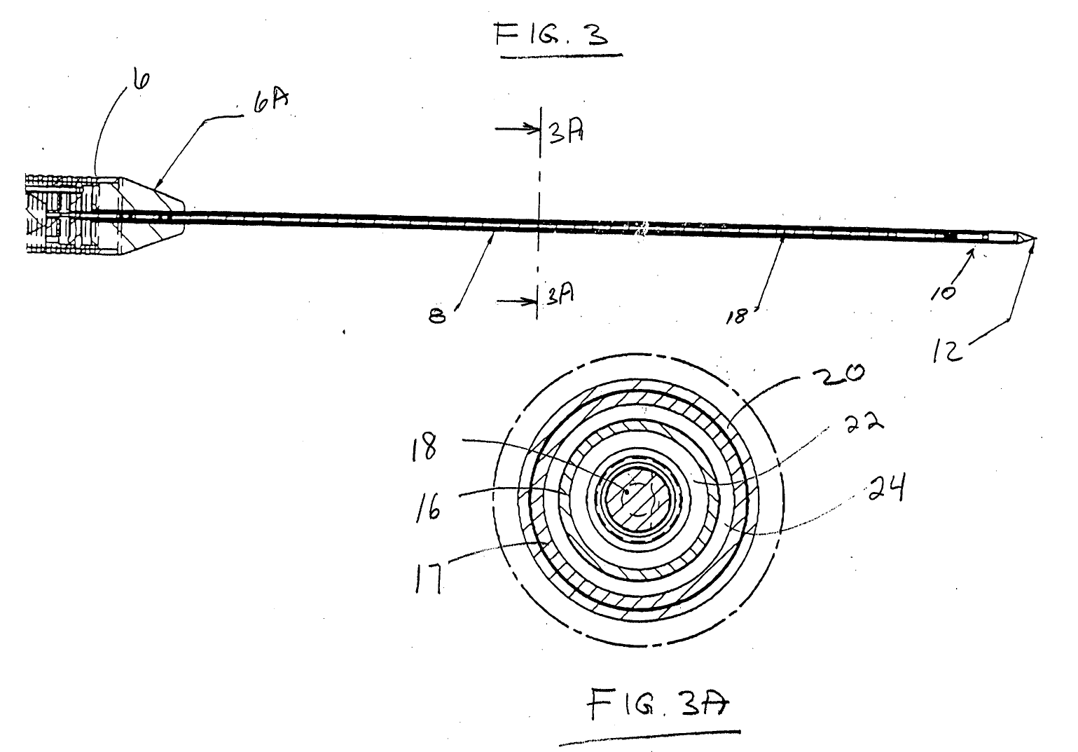

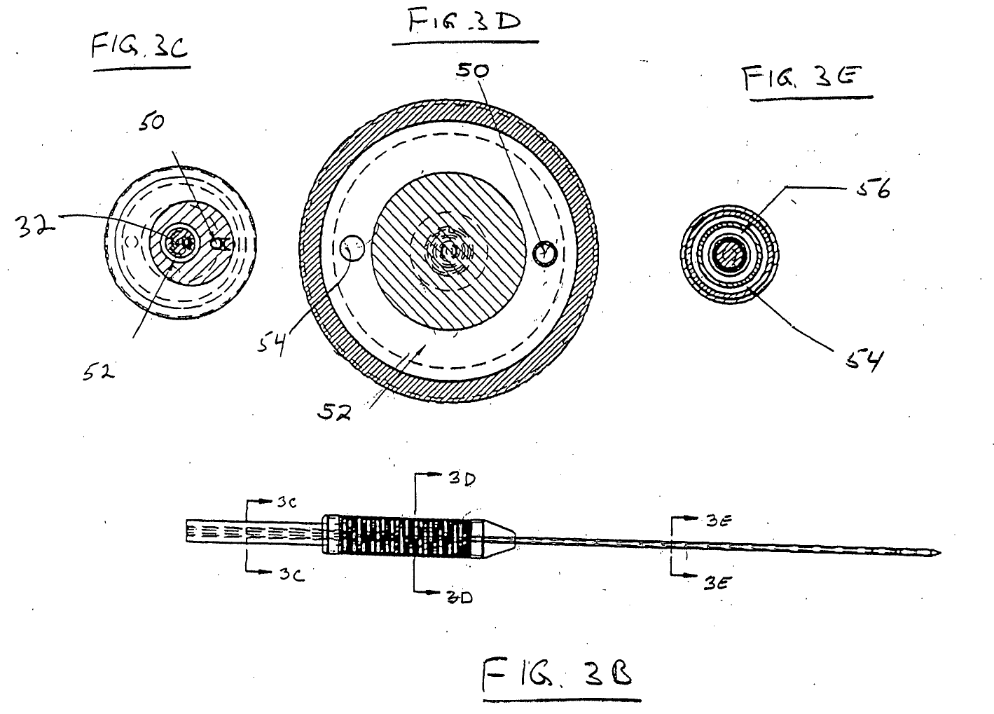

To better understand the present invention the environment of its use is indicated in FIG. 2 which shows the radiating element segment 10 of probe 5 inserted into the central portion 14 of a tumor mass 15 of an organ system 16. To reach the organ system, the probe first had to penetrate the patient's skin unless the organ system was previously exposed.

The new cell necrosis apparatus will now be described in terms of: (a) the probe handle, body and tip, (b) the cable chassis terminating in the microwave antenna, (c) the cooling system and (d) the microwave generator.

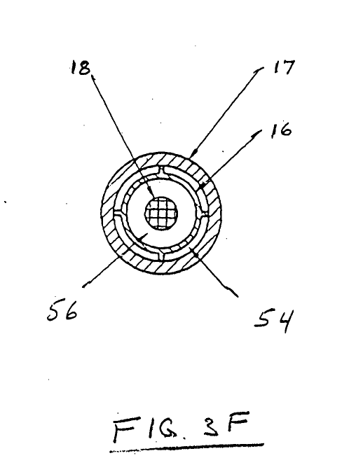

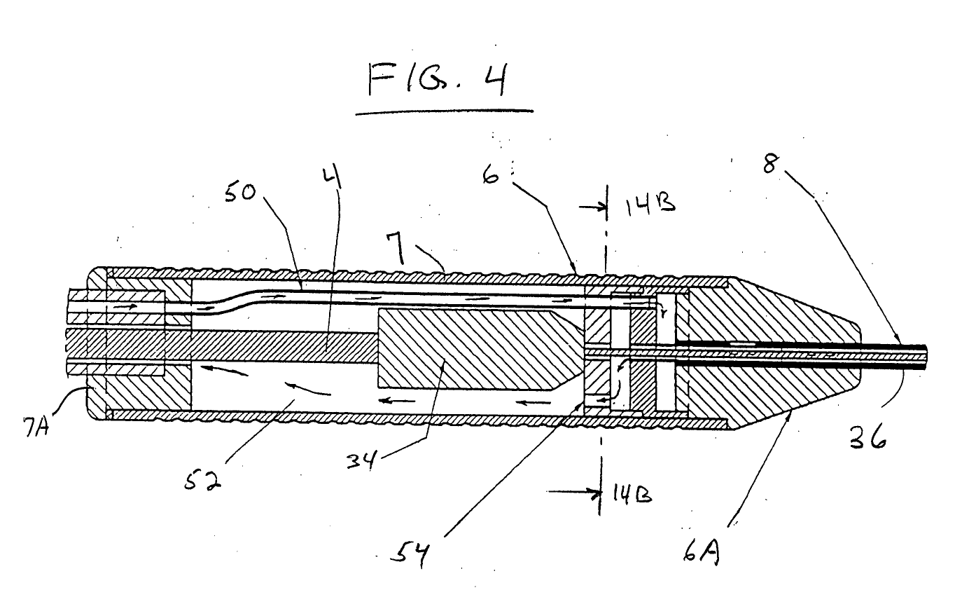

The probe will be described first with reference to FIGS. 1 and 3A-3F, 4 and 5 and other figures as appropriate. As seen in FIGS. 1, 3 and 3A, the probe 5 comprises the handle 6 (shown in part in FIG. 3) with hub 6A at its distal end, probe body 8 formed of transmission line 18 surrounded by coaxial inner and outer lumen tubes 16, 17 and 20. The probe terminates with tip 12. FIG. 3A further shows inlet coolant flow passageway 22 and outlet or return flow passageway 22 defined by said inner and outer lumens which extend along the length of the probe body from the hub 6A to the antenna 10. At the proximal end of the probe body the hub 6A supports both the inner and outer lumens. This hub is mounted to the probe handle. It should be noted that the construction of the probe body is not limited to a metal and plastic composite. The probe body can also be constructed entirely of plastic or high performance carbon fiber tubing.

The diameter of the probe body can range from 7 to 9 Fr in size. For smaller tumor masses the size and exposure lengths of the probe body can be reduced. A typical probe would be 8 Fr in diameter with an exposed length (distance from hub to the tip of the probe) of 20 cm. The probe handle 6 is to be held by the practitioner during placement. The microwave antenna housed within the distal tip of the probe body has a 2.5 cm active length. The transmission line cable is roughly 7 feet in length which allows the practitioner freedom to move the probe without obstruction during placement. The probe has a connector at its proximal end for attachment to a compatible microwave generator with an integral coolant system. Chilled water is circulated throughout the entire probe assembly. A temperature sensor is placed at the distal tip for temperature monitoring after necrosis. The entire probe is disposable.

Regardless of procedure, the proposed device is capable of handling insertion forces because of its adequate construction described later herein. In the probe body the outer lumen tube, which has a circular profile, is constructed of a plastic and metal composite. Non-metallic materials such as plastic must be used to create the radiating element segment, since metal in the field of the radiating element will disrupt the radiation pattern generated by the microwave antenna, which would result in uncontrollable and ineffective cell necrosis. The proximal portion of the outer lumen tube is constructed primarily of metal in order to minimize the potential for buckling of the probe shaft during insertion. Roughly 3.0 cm of the distal portion of the outer lumen is constructed of plastic in order to cover the 2.5 cm active length of the microwave antenna. This plastic tube has a diameter for 3.0 cm along its distal portion, the same as the diameter of the metal lumen. The balance of the plastic tube is ground down to a diameter less than that of its distal portion, thus establishing a step at this junction. The metal lumen is then inserted over the proximal end of the plastic tube so that it abuts against the junction.

The inner lumen 16 is constructed entirely of plastic and resides within the outer lumen 17. Plastics with high flexural moduli such as PEEK (Polyetheretherketone) and PEI (Polyetherimide) are preferred, and various fillers can be added to these resins to further enhance their flexural moduli. The profile of the inner lumen seen in FIG. 3A is circular; however, a splined profile of FIG. 3F can further enhance the overall rigidity and integrity of the probe body and reduce the flexure of the body during loading. To further reduce drag the metallic portion of the outer lumen's surface can be coated with a non-stick surface such as PTFE.

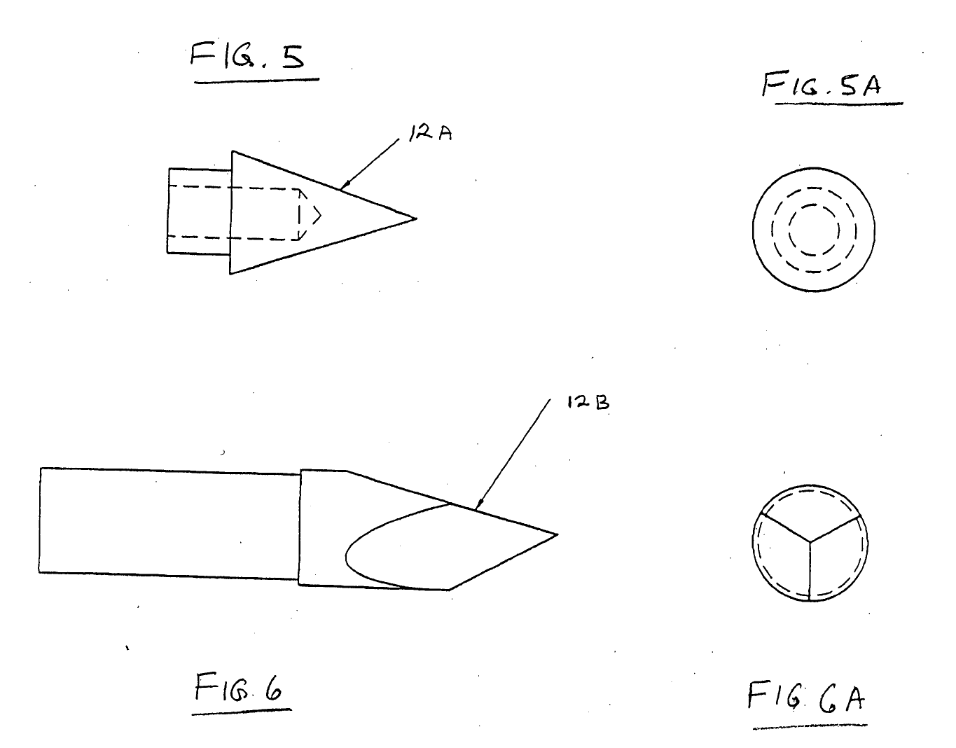

At the distal end of the probe body 5 a tip 12 of plastic or metal is grafted to the outer lumen. Various manufacturing methods can be used to graft the tip including radio frequency energy and the application of medical grade adhesives. To minimize the insertion force of the probe, various tip designs 12A, 12B can be used as illustrated in FIGS. 5, 5A, 6 and 6A.

The probe handle 6 is shown in FIGS. 1-3 and in detail in FIG. 4. This handle has a tubular outer shell 7 that forms a housing closed by plate 7A at the proximal end and by hub 6A at the distal end. Proximal end plate 7A has a central aperture to receive the transmission cable 4 and an offset aperture to communicate with inlet lumen 50 and a coaxial aperture also seen in FIG. 3C, to receive the coolant return flow which then flows along the flexible cable 4 to the coolant reservoir. Within probe handle 6 is splice 34 from which the transmission line 18 extends distally through hub 6A and thence through probe body 8.

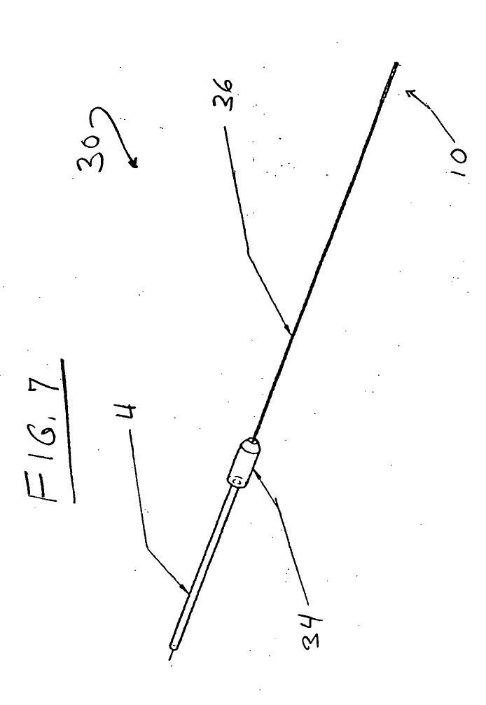

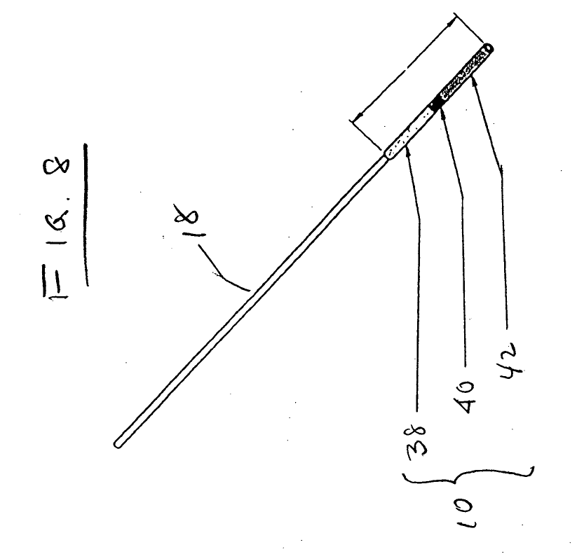

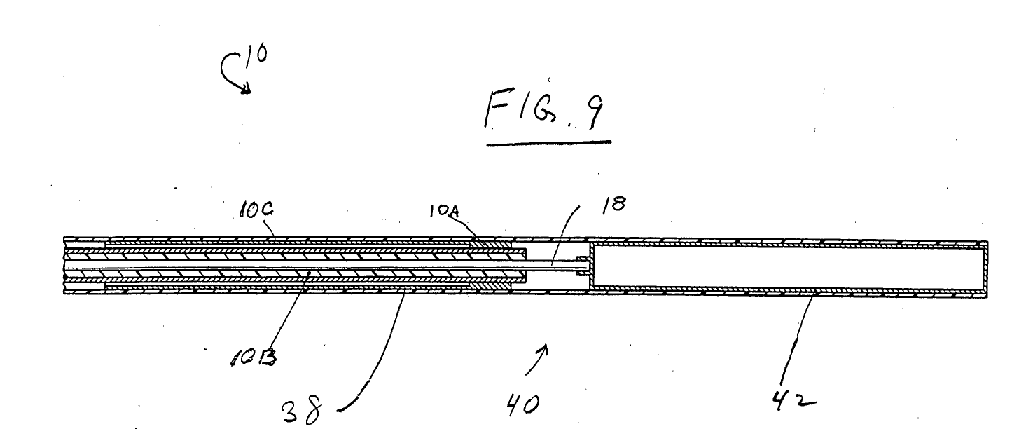

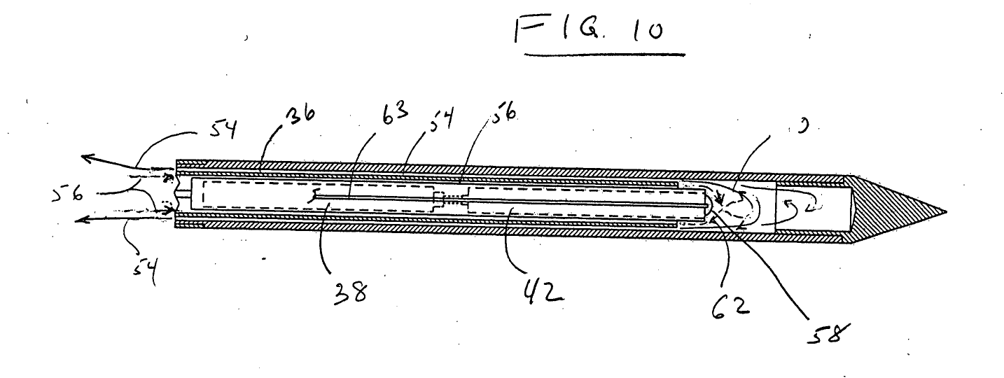

FIG. 7 shows the cable chassis 30 terminating in the antenna 10, and FIGS. 8 and 9 show the cable and antenna construction. The cable chassis comprises flexible coaxial cable feedline 4, hermetically sealed splice 34, semi-rigid cable 36 and radiating elements of antenna 10. As indicated in FIG. 7 and seen in FIG. 4, splice 34 will be situated in probe handle 6, and cable 36 and radiating elements 10 will be situated in probe body 8. FIG. 8 shows the radiating elements comprise proximal element 38 and distal element 42 separated by central element 40.

Referring again to FIG. 7, both of the cables 4 and 36 are provided by Micro-coax of Limerick, Pa. and are also available by various other cable manufacturers. The feedline cable 4 is constructed of a larger gauge flexible coaxial cable such as UFB142C. The hermetically sealed junction 34, also seen in FIG. 4, is used to connect the flexible and semi-rigid cables. The seal is both airtight and watertight so that coolant can be applied over the entire assembly without causing an electrical short. Cable 36 is constructed of a semi-rigid coaxial cable such as UT-34. Typically electrical characteristics for each of these cables include excellent power handling and insertion loss properties. In order to maximize the amount of power delivered to the antenna, the losses through the entire cable chassis need to be minimized. The microwave antenna must be “electrically” matched in order to maximize the depth of the EMF (electromagnetic field) and to minimize additional cable losses. The calculation, as seen in Appendix A attached hereto, illustrates this design determination.

The semi-rigid coaxial cable 36 is coated with a material of low dielectric property such as PTFE, which can be applied via means of spray coating or installed in the form of heat-shrink tubing. The inner conductor of the cable terminates at the distal element of the antenna 10. The proximal element communicates with the distal end of the semi-rigid cable's outer conductor, this junction being formed by means of welding the outer conductor of the semi-rigid coaxial cable to the proximal element. Both the proximal and central elements are constructed of medical grade metal. The central element is constructed of either epoxy or PTFE filler. The entire assembly is insulated with PTFE shrink tubing.

The bipolar choked antenna design allows for minimal insertion losses and optimal performance. With conventional (non-choked) microwave antennas, the radiating performance is a function of insertion depth. For optimal radiation the antenna must have sections that are equal in length and that correspond to a quarter wavelength in tissue. In most cases, this is not clinically practical since insertion depth is a function of the clinical situation. As a result, conventional microwave antennas typically have increased reflected power at the antenna junction, which results in increased input power requirements and ohmic heating of the transmission and antenna feed lines. Moreover, the overall antenna may have unbalanced radiating patterns. A variety of different connectors can be used at the proximal section of the antenna chassis including but not limiting to SMA, N and SMB connectors which are standard in the industry.

The coolant system and pathways are illustrated in FIGS. 3A-3F, 4, and 10. The coolant circulates through probe 5 and is re-circulated within the generator system. Chilled water at about 40° F. is circulated throughout the entire probe assembly for several important reasons. First, chilling allows the antenna and transmission line to operate at higher powers over an extended period of time. Larger burn profiles result at higher wattages along with more time. Second, chilling of the antenna portion allows for a greater depth of penetration. By cooling the outer probe surface around the antenna, the therapeutic heating radius is increased. This is based on maximizing the coolant power in order to minimize the overall power difference of the system. Smaller power differences optimize the amount of radiating energy delivered to the tumor site resulting in larger than normal volumes of necrosis. This power difference is illustrated by the calculations shown in Appendix B attached hereto.

Third, lesions created by microwave antennas typically yield tear drop profiles resulting in tracking. This is caused by conductive energy which tracks proximally beyond the antenna. Cooling eliminates this profile and allows for a more elliptical to spherical lesion with no tracking. Finally, cooling of the probe body allows for patient comfort during the procedure at the entry site. Cooling also allows the practitioner to hold the feedline during the procedure, all of these design features enhancing the capability and controlability of this apparatus and procedure for treating lesions.

The coolant pathways are illustrated in the elevational views of FIGS. 4 and 10 and the sectional views of FIGS. 3A-3F. As seen in FIG. 3, coolant originating with the coolant circulating reservoir and pump 2 flows through return ducts 50, 52 which coaxially surround the flexible feedline 18 in cable 4. FIGS. 4 and 3D show the coolant's inlet duct within the probe handle 6, continuing adjacent and slightly laterally spaced from feedline 4, and the return duct 52 generally surrounding the feedline 4. In FIGS. 4 and 3D the reference numeral 56 refers to a fluid flow return duct. FIGS. 3E and 3F show flow inlet 56 and flow return 54 ducts within the probe body. Obviously, all the segments of the inlet flow passageway in the transmission line, probe handle and probe body are continuous, and all the segments of the return flow passageway are similarly continuous.

FIG. 9 shows an enlarged sectional elevational view of the antenna 10 of FIG. 8, including inner conductor 18 electrically coupled to distal element 42, outer conductor 10A electronically coupled to proximal element 38, inner insulation 10B and outer insulation 10C.

FIG. 10 shows the end portion of the inlet flow passage 56 as it flows along the proximal and distal parts 38, 42 of the antenna, turns, reverses per arrows 60, and then begins its return flow via arrows 62 in the return duct 54 coaxially adjacent the antenna. As noted earlier, the coaxial flow of both the inlet and return coolant along the probe body and about the antenna establish a symmetrical radiation pattern.

A microwave generator, which generates a frequency of either 915 MHz, or 2450 MHz, is connected to the proximal portion of the probe transmission line. Note that the length of the antenna would differ depending on the operating frequency. The generator should have an input power between 30-200 Watts. More than one output channel could be designed into the generator. Hence simultaneous necrosis sites could be generated with multiple devices. The refrigerated coolant and pump system can either be integral to the microwave generator or self-standing. Chilled water maintained at 40° F. and at a flow rate between 60-170 cc/min is preferred. By surface cooling the antenna radiating elements, the treatment volume may be pushed to larger values of radius.

A temperature sensor 58, either thermocouple or fiber optic sensor, is placed at the distal tip of the probe, as seen in FIG. 10. This sensor is attached by means of an adhesive and runs distally by strip 63 along the inner lumen to a connector (not shown). Temperature measuring is normally conducted only when the microwave system along with the coolant pump is turned off and is controlled by the microwave generator. Temperature sensing is possible during the ablation process with more advanced measuring techniques; however, the information may be less useful. Temperature sensing is useful because it allows the practitioner to determine whether the device is working, and also it allows the physician to re-guide the probe into an untreated area by a simple temperature measurement.

An optional sheath (not shown) can be placed over the probe. Such sheath would be connected to the distal portion of the probe delivery systems body by means of a luer lock, which would prevent the sheath from separating from the probe delivery system during the treatment session. The purpose of the sheath is twofold. First it can be used to inject chemicals such as ethanol, acetic acid or saline solution into the target treatment area in order to aid in the treatment process. Second, the sheath can provide a means for tracking the wound site after the procedure. This would allow the practitioner to plug the wound site after the treatment session has been completed and the probe delivery system has been removed from the patient. Typically a practitioner would infuse a fibrin material through the sheath side arm to minimize bleeding from the wound site. The sheath has a valve within its proximal hub to prevent blood and other bodily fluids from leaving the wound site.

Although the present invention has been described with reference to preferred embodiments, persons skilled in the art will recognize that changes may be made in form and detail without departing from the spirit and scope of the invention.

1. A cell necrosis apparatus for delivering thermal microwave energy to a specific site in a body, comprising:
a. a microwave generator,
b. a coolant delivery system for delivering and circulating a quantity of cooled liquid coolant via inlet and return passageways,
c. a probe comprising a probe handle and a probe body having a proximal portion coupled to said probe handle and a distal portion,
d. a microwave antenna in said distal portion of said probe body for applying thermal microwave energy to said specific site in cell necrosis treatment, and
e. a microwave transmission line extending from said microwave generator to and through said probe handle and to and through said probe body to said microwave antenna and electrically coupled thereto,
f. said inlet and return coolant flow passageways extending from said coolant delivery system to and through said probe handle and thence extending coaxially about said antenna and along the length thereof within said probe body, where a first of said inlet and return coolant flow passageways is radially outward of and immediately adjacent said microwave transmission line and said antenna within said probe body and the other of said inlet flow passageways is radially outward of said first flow passageway.

2. A cell necrosis apparatus according to claim 1 wherein said inlet passageway in said probe body is adjacent said transmission line and said antenna and is radially inward of said return passageway in said probe body.

3. A cell necrosis apparatus according to claim 2 wherein said inlet and return passageways extend coaxially in said transmission cable extending from said coolant delivery system to said probe handle and communicate with said inlet and return passageways in said handle respectively.

4. A cell necrosis apparatus according to claim 1 wherein said microwave antenna is a dipole type comprising proximal and distal parts axially spaced by a central part.

5. A cell necrosis apparatus according to claim 1 wherein said microwave transmission line is electrically insulated from said coolant flow in said coolant flow passageways.

6. A cell necrosis apparatus according to claim 1 further comprising temperature sensor means only in the region of said antenna.

7. A cell necrosis apparatus according to claim 6 wherein said microwave antenna has a distal end and said temperature sensing means is located at the distal end of said microwave antenna.

8. A cell necrosis apparatus according to claim 1 wherein said probe further comprises a sharp pointed tip at said distal portion thereof and extending in the distal direction.

9. A cell necrosis apparatus according to claim 1 wherein said probe has an external diameter in the range of 6-10 Fr.

10. A method of cell necrosis treatment with a cell necrosis apparatus including a microwave antenna in a probe, comprising the steps:
a. providing an annular coolant passageway adjacent and circumferentially surrounding said antenna and extending lengthwise and coaxial therewith,
b. providing a source of coolant, inlet and return duct means for communicating said coolant between said source and said antenna, and means for circulating said coolant through said inlet and return duct means, and
c. flowing said coolant in said annular coolant passageway while activating said microwave antenna, thereby producing a symmetrical radiation pattern about said antenna.

11. A method according to claim 10 comprising the further step of forming said annular coolant passageway to extend in the distal direction as an inlet path adjacent said antenna and to extend in the proximal direction as a return path situated radially outward of and coaxial with said inlet path, where said coolant flows from said source first through said inlet path, then through said return path and finally back to said source.

12. A method of cooling the antenna of a microwave cell necrosis apparatus which includes a probe handle and a probe body having a proximal portion coupled to said probe handle, a microwave antenna in said distal portion of said probe body for applying thermal microwave energy in cell necrosis treatment, a microwave transmission line extending from a microwave generator to and through said probe handle and to and through said probe body to said distal portion thereof and electrically coupled to said microwave antenna, said method comprising the steps:
a. providing a coolant source and coolant delivery apparatus,
b. establishing inlet and return coolant flow passageways extending from said coolant delivery apparatus to and through said probe handle and thence extending coaxially about said microwave transmission line and along the length thereof within said probe body, and extending coaxially about said antenna and along the length thereof within said probe body, where a first of said inlet and return coolant flow passageways is radially outward of and immediately adjacent said microwave transmission line and antenna, and the other of said inlet flow passageways is radially outward of said first flow passageway, and
c. circulating said coolant from said source of coolant via said flow passageways to and about said antenna along the length thereof.

13. A method according to claim 12 wherein said inlet passageway in said probe body is immediately adjacent said transmission line and said antenna and is radially inward of said return passageway in said probe body.

14. A method according to claim 10 for creating a necrosis area within a patient's organ being treated, said method being operable with a microwave generator electrically coupled to said antenna and a water pump means for controlling flow of said coolant through said inlet and return duct means.

15. A method according to claim 14, wherein said microwave generator generates a frequency of either 915 MHz or 2450 MHz.

16. A method according to claim 14, wherein input power to said microwave generator, time of operation and/or coolant flow rate are selectable by a user.

17. A method according to claim 14 wherein said input power is in the range of about 10 to 200 watts.

18. A method according to claim 16 wherein said time of operation is in the range of about 1 to 20 minutes.

19. A method according to claim 16 when said coolant flow rate is in the range of about 60-170 cc/min.

20. A method according to claim 10 wherein said coolant that flows through said coolant passageway is maintained at a temperature of about 40° F.

21. A method according to claim 15 for creating a necrosis of about 4-5 cm in diameter and about 5.5 cm in length, by operating a 2450 MHz microwave generator at about 200 watts of input power for about 15 minutes.

22. A method according to claim 15 for creating a necrosis of about 2-3 cm in diameter and about 3.5 cm in length, by operating a 2450 MHz microwave generator at about 200 watts of input power for about 10 minutes.

23. Apparatus according to claim 1 wherein said microwave generator generates a frequency of either 915 MHz or 2450 MHz, with an input power range of 10-200 watts.

24. Apparatus according to claim 1 wherein said coolant delivery system causes a coolant flow rate in the range of 60-170 cc/min.

25. Apparatus according to claim 1 wherein said inlet and return passageways are dimensioned for cooperation with said coolant delivery system to cause a coolant flow rate in the range of 60-170 cc/min.

26. Apparatus according to claim 1 wherein said probe has diameter of range of about 2-5 cm and length in the range of about 3.5-5.5 cm.
